(19) United States
(12) Patent Application Publication
(10) Pub. No.: US 2012/0148538 A1
(43) Pub. Date: Jun. 14, 2012

(54) COMPOSITION FOR HARD TISSUE FORMATION AND, DENTIN OR PULP REGENERATION CONTAINING AMELOBLAST, APICAL BUD CELL OR ITS CULTURE FLUID AS AN ACTIVE INGREDIENT

(75) Inventors: Joo-Cheol PARK, Seoul (KR); Won-Jun SHON, Seoul (KR); Jihyun LEE, Seoul (KR)

(73) Assignee: SNU R&DB FOUNDATION, Seoul (KR)

(21) Appl. No.: 13/182,011

(22) Filed: Jul. 13, 2011

(30) Foreign Application Priority Data
Dec. 13, 2010 (KR) KR10-2010-0127022
Jul. 13, 2011 (KR) KR10-2011-0069292

(51) Int. Cl. A61K 35/32; A61P 19/08; C12N 5/071

(52) U.S. Cl. 424/93.7; 424/115; 435/366

(57) ABSTRACT

The present invention relates to a composition for hard tissue formation and dentin, or dental pulp regeneration comprising ameloblasts, apical bud cells, or its culture fluid as an active ingredient. The ameloblasts, apical bud cells, or its culture fluid promotes the differentiation of odontoblasts, human dental pulp stem cells and osteoblasts, increases DSPP promoter activity, induces dental pulp regeneration of dental pulp stem cells, and induces hard tissue formation significantly in vivo, so that they can be effectively used for a composition for promoting hard tissue formation and dentin or dental pulp regeneration.

### CROSS-REFERENCES TO RELATED APPLICATION

This patent application claims the benefit of priority from Korean Patent Application No. 10-2010-0127022, filed on Dec. 13, 2010 and No. 10-2011-0069292, filed on Jul. 13, 2011, the contents of which are incorporated herein by reference.

### BACKGROUND OF THE INVENTION

1. Field of the Invention

The present invention relates to a composition for hard tissue formation and dentin or dental pulp regeneration, more precisely a composition for hard tissue formation and dentin or dental pulp regeneration using ameloblasts, apical bud cells, or its culture fluid as an active ingredient.

2. Description of the Related Art

Odontoblasts, being attached to dentin, are columnar cells forming dentin (Victor, E.; Arana-Chavez, V. E.; Massa, L F. OdontOblasts: the cells forming and maintaining dentine. The International Journal of Biochemistry & Cell Biology, 2004 36: 1367-73. The final differentiation of odontoblasts is regulated by the ectoderm derived inner dental epithelium. It has been known so far that a variety of factors are involved in the differentiation of odontoblasts, which are exemplified by various growth factors secreted in basement membrane and enamel knot, DMPI, and DMP4 (Lisi S, Peterkov R, Peterka M, Vonesch J L, Ruch J V, Lesot H. Tooth morphogenesis and pattern of odontoblast differentiation. Connect Tissue Res. 2003; 44, 1:167-70; Smith A J, Lesot H. Induction and regulation of crown dentinogenesis: embryonic events as a template for dental tissue repair? Crit. Rev Oral Biol Med. 2001; 12:425-37). Laminin alpha2, one of constituents of basement membrane, regulates DSPP expression during the differentiation of odontoblasts (Yuasa K, Fukumoto S, Kamasaki Y, Yamada A, Fukumoto E, Kanaoka K, Saito K, Harada H, Arikawa-Hirasawa E, Miyagoe-Suzuki Y, Takeda S, Okamoto K, Kato Y, Fujiwara T. Laminin alpha2 is essential for odontoblast differentiation regulating dentin sialoprotein expression. J Biol. Chem. 2004 Mar. 12; 279(11):10286-92). A. George et al reported that DMPI and DMP4 regulate the differentiation of odontoblasts (Narayanan, K.; Srinivas, R.; Ramachandran, A. Nao, J.; Quinn, B. George, A. Differentiation of embryonic mesenchymal cells to odontoblast-like cells by overexpression of dentin matrix protein 1. PNAS, 2001, 98 (8), 14516-21; Narayanan, K.; Gajjeraman, S.; Ramachandran, A.; Hao, J.; George, A. Dentin matrix protein 1 regulates dentin sialophosphoprotein gene transcription during early odontoblast differentiation. J Biol. Chem. 2006, 281(28), 19064-71; Hao, J.; Narayanan, K.; Muni, T.; Ramachandran, A.; George, A. Dentin Matrix Protein 4, a Novel Secretory Calcium-binding Protein That Modulates Odontoblast Differentiation. J Biol. Chem. 2007, 282(21), 15357-65). It has been also reported that the growth factor of enamel knot could regulate the differentiation of odontoblasts (Thesleff, I.; Ker, S. V.; Jernvall, J. Enamel Knots as Signaling Centers Linking Tooth Morphogenesis and Odontoblast Differentiation. Adv. Dent. Res., 2001, 15, 14-8). It has recently been suggested that insulin growth factor, retinoic acid, epithermal growth factor, and neuronal growth factor are also the factors inducing odontoblast differentiation (Lesot, H.; Lisi, S.; Peterkova, R.; Peterka, M.; Mitolo, V.; Ruch, J. V. Epigenetic Signals during Odontoblast Differentiation. Adv. Dent. Res., 2001, 15, 8-13; Arany, S.; Koyota, S.; Sugiyama, T. Nerve growth factor promotes differentiation of odontoblast-like cells. J Cell Biochem. 2009, 106(4), 539-45). However, those factors involved in odontoblast differentiation including DMPI and DMP4 are not the ones secreted specifically in epithelial cells. Therefore, based on the theory that inner dental epithelium regulates the final step of odontoblast differentiation, it is necessarily investigated to identify a factor inducing odontoblast differentiation from ameloblasts originated from inner dental epithelium and its products.

Human dental pulp stem cells (hDPSCs) are easy to isolate from human third molar, which are differentiated into odontoblasts and osteoblasts, and have the similar characteristics to mesenchymal stem cells (d'Aquino R, De Rosa A, Laino G, Caruso F, Guida L, Rullo R, Checchi V, Laino L, Tirino V, Papaccio G., Human dental pulp stem cells: from biology to clinical applications., J Exp Zool B Mol Dev Evol. 2009 Jul. 15; 312B (5):408-15). Gronthos, et al had succeeded in isolation of human dental pulp stem cells and then demonstrated that the differentiation of the dental pulp stem cells into odontoblasts, chondrocytes, adipocytes, and nerve cells could be possible (Gronthos S, Mankani M, Brahim J, Robey P G, Shi S. Postnatal human dental pulp stem cells (DPSCs) in vitro and in vivo. Proc Natl Acad Sci USA. 2000 Dec. 5; 97(25):13625-30; Iohara K, Meng L, Ito M, Tomokiyo A, Matsushita K, Nakashima M. Side population cells isolated from porcine dental pulp tissue with self-renewal and multipotency for dentinogenesis, chondrogenesis; adipogenesis, and neurogenesis. Stem Cells. 2006 November; 24(11):2493-503. Epub 2006 July 27).

Dental pulp is a loose connective tissue supporting dentin, which is composed of cells, extracellular matrix, blood vessels, nerve, and lymph ducts. Dental pulp is involved in the maintenance, support and continuous formation of dentin. Dental pulp has sensory function, and delivers nutrition to dentin, and provides protection for pulp by forming tertiary dentin (Histogenesis. Oral Tissues., 145-162, Park J C, et al., Komoonsa). However, the conventional method to treat dentin-pulp disease, mainly focused on the conservation of dentin-pulp, has disadvantages of weakening of treated teeth and high chance of re-infection (Kim J Y, Xin X, Moioli E K, Chung J, Lee C H, Chen M, Fu S Y, Koch P D, Mao J J., Regeneration of Dental-Pulp-like Tissue by Chemotaxis-Induced Cell Homing., Tissue Eng Part A., 2010, Jun. 24; Murray P E, Garcia-Godoy F, Hargreaves K M., Regenerative endodontics: a review of current status and a call for action., J. Endod., 2007, April; 33(4):377-90). There have been many attempts to generate dental pulp like tissues in previous studies. And some factors such as DMPI, FGF, and BMP7 have been approved to be, effective in dental pulp regeneration. But, in the treatment of these factors, normal arrangement of odontoblasts on the surface of dentin has not been observed at all (Prescott R S, Alsanea R, Fayad M I, Johnson B R, Wenckus C S, Hao J, John A S, George A, In vivo generation of dental pulp-like tissue by using dental pulp stem cells, a collagen scaffold, and dentin matrix protein 1 after subcutaneous transplantation in mice., J. Endod. 2008 April; 34(4):421-6).

Rodent incisors have a special epithelial structure, referred to as the ‘apical bud,’ at the apical end, which continuously grows. The apical bud contains inner enamel epithelium, outer enamel epithelium, and stellate reticulum, and the inner enamel epithelium differentiates into ameloblasts (Harada H, Ohshima H. New perspectives on tooth development and the dental stem cell niche. Arch Histol Cytol 2004 March; 67(1):1-11; Ohshima H, Nakasone N, Hashimoto E, Sakai H, Nakakura-Ohshima K, Harada H. The eternal tooth germ is formed at the apical end of continuously growing teeth. Arch Oral Biol. 2005 February; 50(2):153-7. Epub 2004 November 23). Jin Y et al have recently reported that dental pulp cells were differentiated into odontoblasts when epithelial and mesenchymal cells were co-cultured using tooth germ conditioned medium (Yu, J.; Deng, Z.; Shi, J.; Zhai, H.; Nie, X.; Zhuang, H.; Li, Y.; Jin, Y. Differentiation of dental pulp stem cells into regular-shaped dentin-pulp complex induced by tooth germ cell conditioned medium. Tissue Eng., 2006, 12(11), 3097-105; Yu J, Wang Y, Deng Z, Tang L, Li Y, Shi J, Jin Y. Odontogenic capability: bone marrow stromal stem cells versus dental pulp stem cells. Biol Cell. 2007 August; 99(8):465-74). It was additionally reported that when dental pulp cells were transplanted together with apical bud cells, enamel-dentin complex was formed (Yu, J.; Jin, F.; Deng, Z.; Li, Y.; Tang, L.; Shi, Jin, Y. Epithelial-mesenchymal cell ratios can determine the crown morphogenesis of dental pulp stem cells. Stem Cells Dev., 2008, 17(3), 475-82). These results suggest that epithelium derived factors play an important role in odontoblast differentiation. In particular, Honda et al reported that when epithelial and mesenchymal cells were sequentially seeded, tooth-like structure was formed (Honda, M. J.; Tsuchiya, S.; Sumita, Y.; Sagara, H.; Ueda, M. The sequential seeding of epithelial and mesenchymal cells for tissue-engineered tooth regeneration. Biomaterials, 2007, 28(4), 680-9). That is, epithelial-mesenchymal interactions are important and necessary for dentinogenesis. However, it has not been identified specifically which factor of epithelium can induce the differentiation of odontoblasts.

Recently, proteomic methods have been widely applied to various studies. For example, mineralized tissues, such as dentin and enamel, are used in proteomic analysis. Proteomic analysis reports have been made for enamel and ameloblasts. However, proteomic research has not been performed on secretory factors of ameloblasts (Park E S, Cho H S, Kwon T G, Jang S N, Lee S H, An C H, Shin H I, Kim J Y, Cho J Y. Proteomics analysis of human dentin reveals distinct protein expression profiles. J Proteome Res. 2009 March; 8(3):1338-46; Mangum J E, Veith P D, Reynolds E C, Hubbard M J. Towards second-generation proteome analysis of murine enamel-forming cells. Eur J Oral Sci. 2006 May; 114 Suppl 1:259-65; discussion 285-6, 382).

Thus, the present inventors studied the effect of epithelial cells on the differentiation of odontoblasts to develop a novel composition for hard tissue formation and dentin or dental pulp regeneration. As a result, the present inventors completed this invention by confirming that ameloblasts, apical bud cells, or its culture fluid could accelerate the differentiation of odontoblasts and osteoblasts, increase DSPP promoter activity, and induce regeneration of dental pulp of dental pulp stem cells, and at the same time form a huge amount of hard tissues in vivo, so it can be used effectively as an active ingredient for the composition for hard tissue formation and dentin or dental pulp regeneration.

### SUMMARY OF THE INVENTION

It is an object of the present invention to provide a composition for hard tissue formation and dentin or dental pulp regeneration comprising ameloblasts, apical bud cells, or its culture fluid as an active ingredient.

To achieve the above object, the present invention provides a method for dentin or dental pulp regeneration containing the step of administering a pharmaceutically effective dose of ameloblasts, apical bud cells, or its culture fluid to a subject.

The present invention also provides a method for promoting hard tissue formation containing the step of administering a pharmaceutically effective dose of ameloblasts, apical bud cells, or its culture fluid to a subject.

The present invention further provides a pharmaceutical composition for dentin or dental pulp regeneration comprising ameloblasts, apical bud cells, or its culture fluid as an active ingredient.

In addition, the present invention provides a pharmaceutical composition for promoting hard tissue formation comprising ameloblasts, apical bud cells, or its culture fluid as an active ingredient.

### ADVANTAGEOUS EFFECT

The ameloblasts, apical bud cells, or its culture fluid of the present invention induces the differentiation of odontoblasts and osteoblasts, increases DSPP promoter activity, promotes dental pulp regeneration, and induces hard tissue formation significantly in vivo, so that it can be effectively used for a composition for promoting hard tissue formation and dentin or dental pulp regeneration.

### BRIEF DESCRIPTION OF THE DRAWINGS

The application of the preferred embodiments of the present invention is best understood with reference to the accompanying drawings, wherein:

FIG. 1 is a diagram illustrating the co-culture method of odontoblasts, dental pulp stem cells, osteoblasts, and apical bud cells:

ABCs: apical bud cells;

MDPC-23: mouse odontoblast cell line;

hDPSCs: human dental pulp stem cells; and

MG63: human osteoblast cell line.

FIG. 2 is a set of photographs illustrating the differentiation of apical bud cells into ameloblasts:

A: microphotograph of separated apical bud cells; and

B: photograph showing the expressions of ameloblastin and amelogenin mRNA.

FIG. 3 is a set of graphs and photographs illustrating the promotion effect of apical bud cells on the differentiation of odontoblasts:

control: the MDPC-23 only group;

co-culture: the group co-cultured MDPC-23 and apical bud cells;

A: a graph showing the relative expression level of DSPP mRNA;

B: a graph showing the relative expression level nestin mRNA;

C: a graph showing the relative expression level of BSP mRNA;

D: a graph showing the comparison of proliferation between the control and the co-culture group;

E: a graph showing the quantitative analysis of type I collagen protein expression;

F: a graph showing the quantitative analysis of DSP protein expression; and

G: a set of photographs showing the expressions of DSP, BSP and type I collagen.

FIG. 4 is a set of graphs and photographs illustrating the effects of apical bud cells (ABCs) and dental pulp stem cells (hDPSCs) on the promotion of differentiation:

(control)hDPSCs: the dental pulp stem cells only group; and

(co-culture)hDPSCs+ABCs: the group co-cultured dental pulp stem cells and apical bud cells.

FIG. 5 is a set of graphs and photographs illustrating the effects of ameloblasts (apical bud cells) on the promotion of osteoblast differentiation:

control: the osteoblasts only group;

co-culture: the group co-cultured ameloblasts(apical bud cells) and osteoblasts;

A: a graph showing the relative expression level of Runx2 mRNA;

B: a graph showing the relative expression level of osterix mRNA;

C: a graph showing the relative expression level of type I collagen mRNA;

D: a graph showing the relative expression level of ALP mRNA;

E: a set of photographs showing the expression of BSP protein; and

F: a graph showing the quantitative analysis of BSP protein expression in the control and the co-culture group.

FIG. 6 is a set of graphs and photographs illustrating the changes of DSPP and BSP promoter activity induced by apical bud cells culture fluid:

A: DSP promoter activity;

B: BSP promoter activity;

C: calcium accumulation confirmed by alizarin red S staining;

PA-CM: apical bud cells culture fluid; and

KB-CM: oral epithelial cells culture fluid.

FIG. 7 is a set of photographs illustrating the expressions of stem cell markers confirmed by fluorescent microscope.

FIG. 8 is a set of photographs and a graph illustrating the effect of apical bud cells culture fluid on hard tissue formation.

hDPSCs: the dental pulp stem cells only group (A and D);

hDPSCs+BMP-2: the BMP-2-treated dental pulp stem cells group (B and E);

hDPSCs+PA-CM: the PA-CM-treated dental pulp stem cells group (C and F); and

G: a graph showing the hard tissue formations of hDPSCs, hDPSCs+BMP-2, and hDPSCs+PA-CM.

FIG. 9 is a set of photographs illustrating the effects of apical bud cells culture fluid on the promotion of dentin-pulp complex like structure and odontoblasts:

A: the dental pulp stem cells (hDPSCs) only group;

B: the rhBMP-2-treated dental pulp stem cells group, arrows indicate the cells entrapped in mineralized matrix;

C: the apical bud cells culture fluid-treated dental pulp stem cell group, black arrows indicate odontoblast processes of differentiating odontoblasts; and

D: a scanning electron micrograph (SEM) showing the differentiated odontoblasts in the apical bud cells culture fluid-treated dental pulp stem cells group, white arrows indicate odontoblast processes of differentiating odontoblasts.

FIG. 10 is a set of photographs illustrating the characteristics of hard tissue formed by the apical bud cells culture fluid treated human dental pulp stem cells:

hDPSCs: the dental pulp stem cells only group;

hDPSCs+BMP-2: the BMP-2-treated dental pulp stem cells group; and

hDPSCs+PA-CM: the PA-CM-treated dental pulp stem cells group.

FIG. 11 is a set of photographs illustrating the formation of dental pulp by the treatment of apical bud cells culture fluid in human dental pulp stem cells on oral environment (in tooth slices).

FIG. 12 is a set of photographs illustrating the characteristics of the dental pulp formed by the treatment of apical bud cells culture fluid in human dental pulp stem cells on oral environment (in tooth slices).

FIG. 13 is a set of SEM photographs illustrating the group of dental pulp stem cells treated with apical bud cells culture fluid in tooth slices;

hDPSCs: the dental pulp stem cells only group.

FIG. 14 is a set of photographs and a graph illustrating the DSPP promoter activity after noggin treatment in apical bud cells culture fluid and the result of Western blotting with odontoblasts treated with periostin-neutralized conditioned medium:

A: DSPP promoter activity after noggin treatment in apical bud cells culture fluid; and

B: result of Western blotting with odontoblasts treated with periostin-neutralized conditioned medium.

FIG. 15 is a set of diagrams and a graph illustrating the result of LC-MSMS analysis with apical bud cells culture fluid.

### DESCRIPTION OF THE PREFERRED EMBODIMENTS

Hereinafter, the present invention is described in detail.

The present invention provides a method for dentin or dental pulp regeneration containing the step of administering a pharmaceutically effective dose of ameloblasts, apical bud cells, or its culture fluid to a subject.

The present invention also provides a pharmaceutical composition for dentin or dental pulp regeneration comprising ameloblasts, apical bud cells, or its culture fluid as an active ingredient.

The ameloblasts herein are preferably ameloblast-lineage cells, but not always limited thereto.

The dentin or dental pulp herein is preferably dentin-pulp complex, but not always limited thereto.

The culture fluid herein is preferably the one cultured for 0-10 days from the beginning of differentiation, and more preferably the one cultured for 3-7 days, and most preferably cultured for 3 days.

The apical bud cells culture fluid herein is preferably prepared by the following steps, but not always limited hereto.

1) culturing mouse primary apical bud cells (ABSs) until reaching 90% of confluence;

2) culturing the cells in the medium supplemented with ascorbic acid and i-glycerophosphate;

3) collecting the medium after 3 days in culture;

4) filtering the medium; and

5) precipitating proteins in the medium, followed by eliminating salts therein.

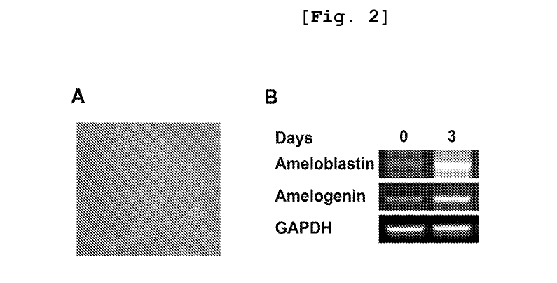

In a preferred embodiment of the present invention, differentiation of apical bud cells into ameloblasts was investigated. As a result, the expression of ameloblast differentiation markers by apical bud cells was confirmed after 3 days in culture (see FIG. 2).

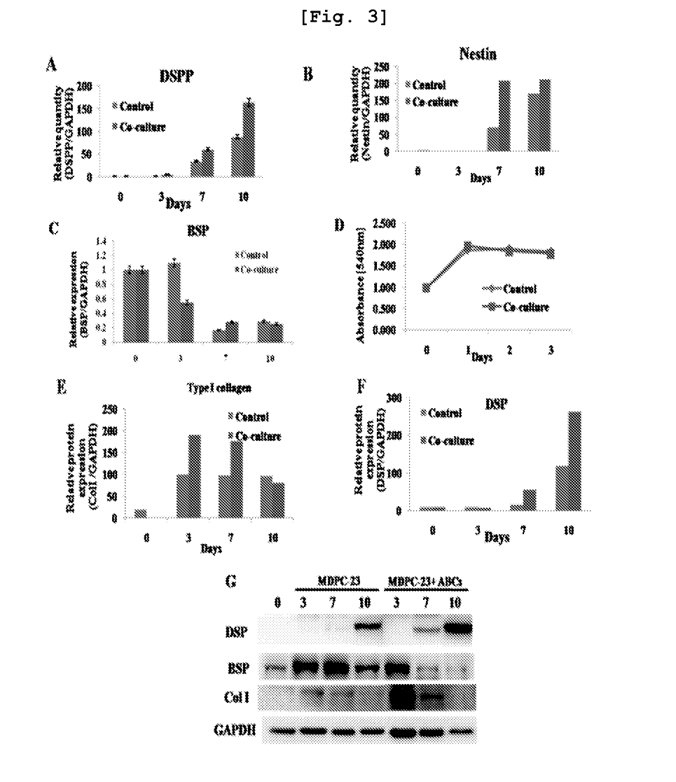

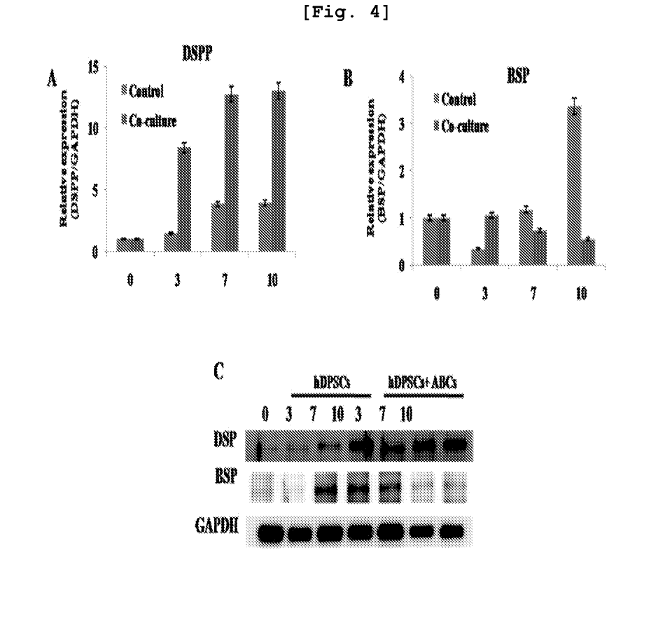

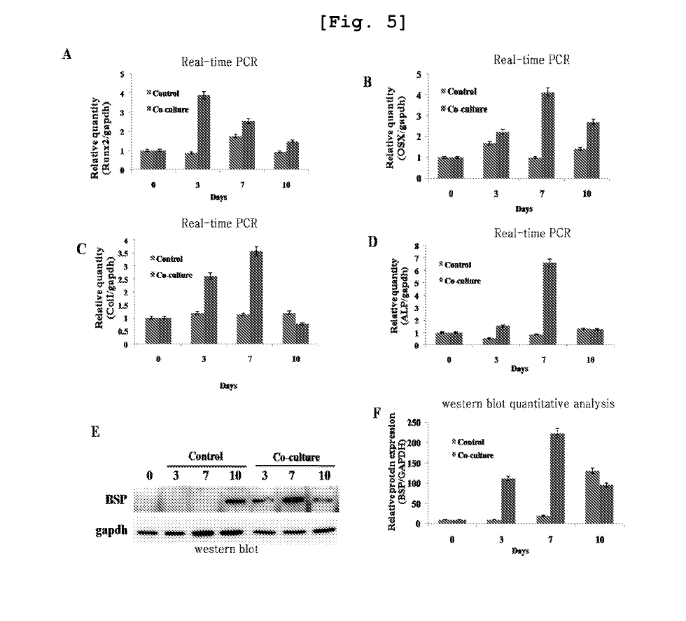

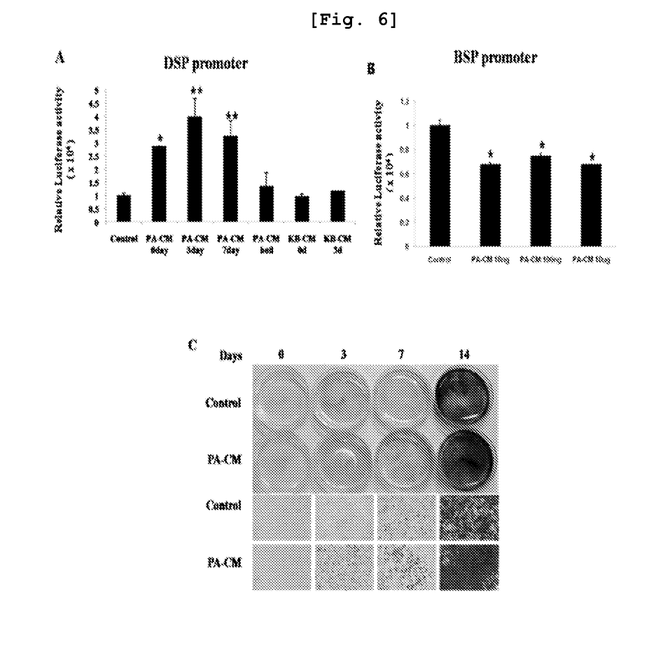

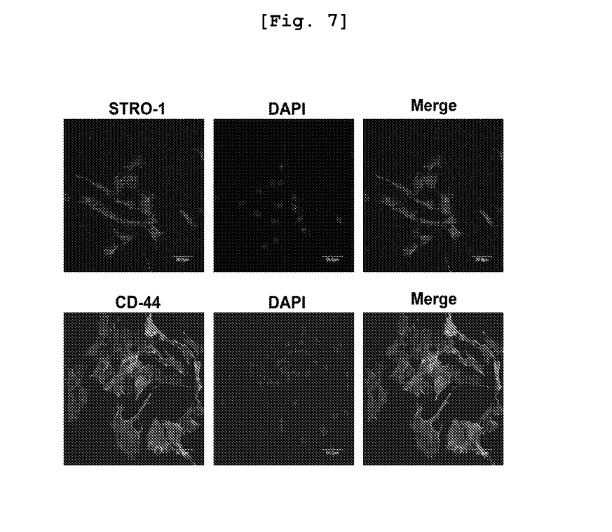

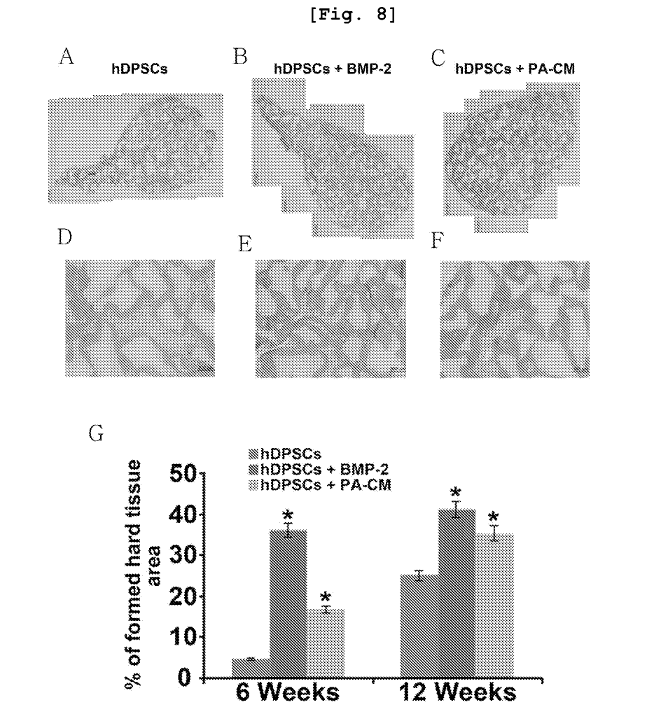

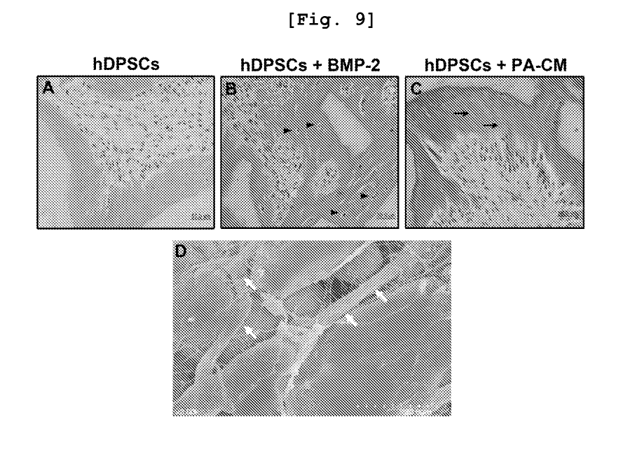

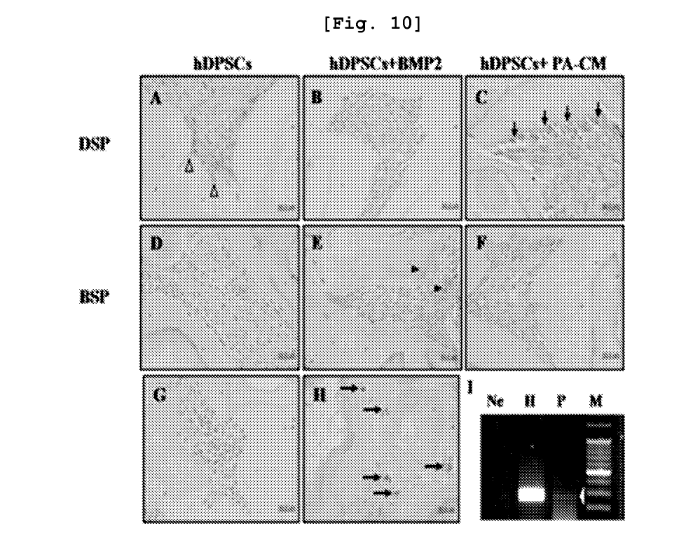

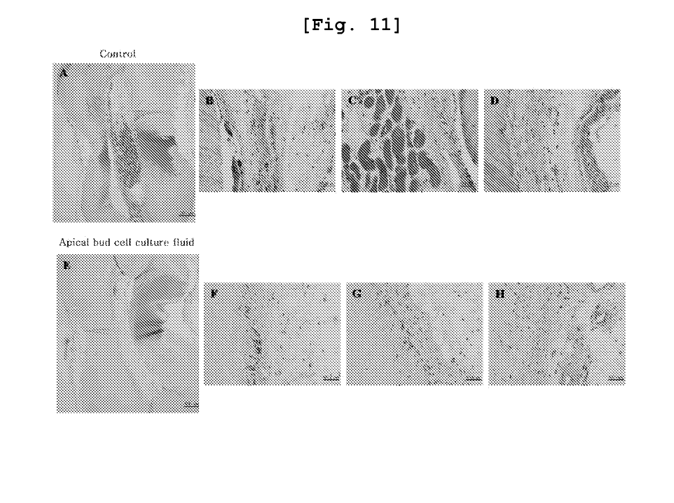

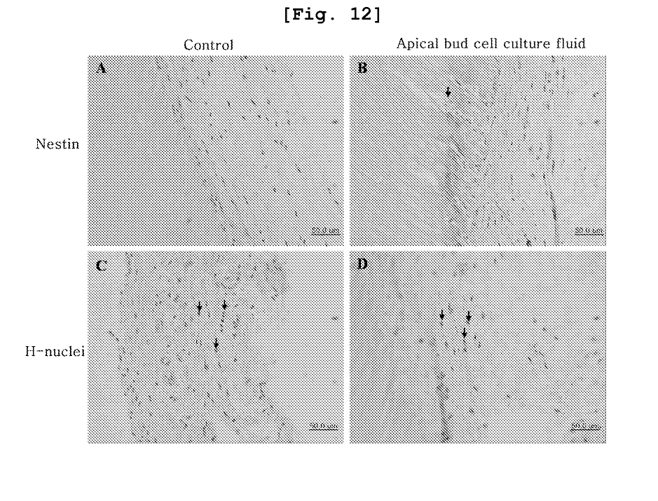

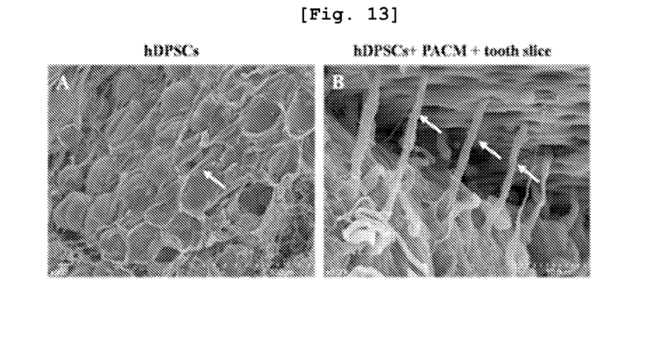

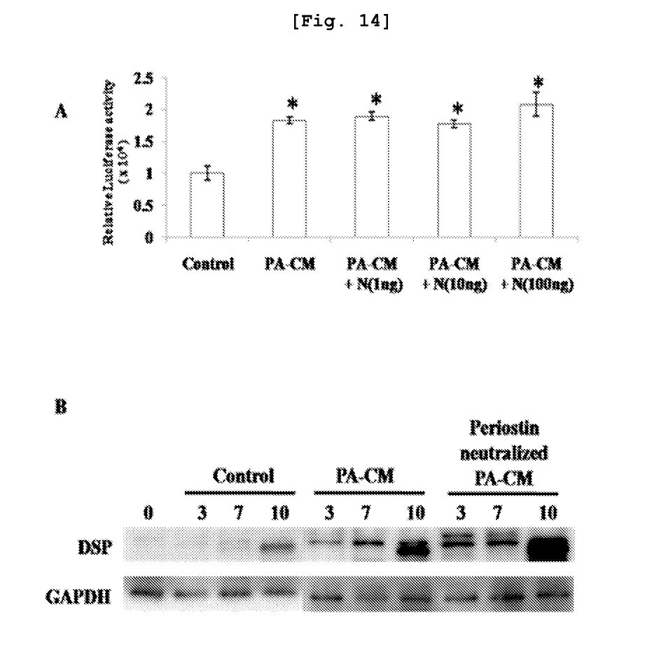

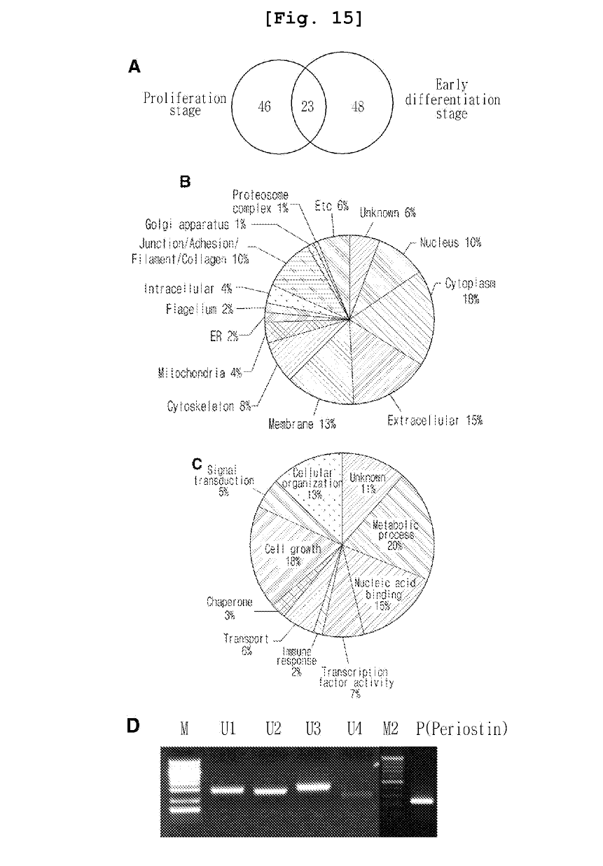

In a preferred embodiment of the present invention, it was confirmed that differentiated odontoblasts secreted DSPP, the noncollageneous protein playing an important role in dentin formation. To confirm the increase of DSPP expression in odontoblasts, apical bud cells were co-cultured with odontoblasts. As a result, it was confirmed that the expression of DSPP was almost double the amount (see FIG. 3A).

In a preferred embodiment of the present invention, it was investigated whether nestin expressed in differentiated odontoblasts was increased. First, apical bud cells were co-cultured with odontoblasts, followed by real-time PCR. As a result, it was confirmed that nestin level was increased 4 times as high as that in the control (see FIG. 3B).

In a preferred embodiment of the present invention, it was investigated whether or not DSP protein forming DSPP and type I collagen, the most abundant collagenous protein in dentin, were increased. To do so, apical bud cells were co-cultured with odontoblasts, followed by Western blotting. As a result, it was confirmed that the expressions of those proteins were increased in experimental groups, compared with those of control (see FIG. 3 E-G). To check the effect of apical bud cells on proliferation of MDPC-23 cells, MTT assay was carried out. The proliferation of MDPC-23 cells showed no differences between control and co-culture (See FIG. 3D).

In a preferred embodiment of the preset invention, it was investigated whether apical bud cells induced odontoblast differentiation of dental pulp stem cells. To do so, dental pulp stem cells were co-cultured with apical bud cells. As a result, the expression level of DSPP, marker differentiated odontoblasts, increased in dental pulp stem cells co-cultured with apical bud cells (see FIG. 4).

In a preferred embodiment of the present invention, it was investigated whether dental pulp stem cells could induce regeneration of dental pulp when apical bud cells culture fluid was treated. First, apical bud cells culture fluid was treated in tooth-like environment. As a result, it was confirmed that pulp-like tissues were formed (see FIG. 11).

In a preferred embodiment of the present invention, the effect of apical bud cells culture fluid on DSPP promoter activity was investigated. The promoter activity was measured by using various apical bud cells culture fluids. As a result, DSPP promoter activity was increased in all apical bud cells culture fluids. Particularly, apical bud cells culture fluid after 3 days in culture was most effective in increasing DSPP promoter activity (see FIG. 6).

In a preferred embodiment of the present invention, it was investigated if the confirmed characteristics above were unique ones of epithelium. To do so, KB cells (oral epithelial cells) were used for the same experiment as above. As a result, KB cells culture fluid had no influence on DSPP promoter activity (see FIG. 6 A and FIG. 6B). Apical bud cells culture fluid was treated to odontoblasts and then calcium accumulation was measured. As a result, calcium accumulation was increased, compared with that in the control (see FIG. 6C).

In a preferred embodiment of the present invention, proteomic analysis was performed to investigate what type of protein is contained in apical bud cells culture fluid. As a result, 113 proteins were identified (see FIG. 15).

In a preferred embodiment of the present invention, the effect of periostin confirmed by western blotting on apical bud cells culture fluid was investigated. As a result, periostin-neutralization in apical bud cells culture fluid did not alter the effects of apical bud cells culture fluid on the odontoblast differentiation (see FIG. 14).

Therefore, it was confirmed from the co-culture of ameloblasts, apical bud cells, or its culture fluid with odontoblasts that the expressions of DSPP and nestin were increased and at the same time the formations of DSP and type I collagen were increased as well. Moreover, apical bud cells culture fluid had the effect of inducing DSPP promoter activity, inducing dental pulp regeneration, and increasing calcium accumulation. Thus, the said ameloblasts, apical bud cells, or its culture fluid can be effectively used as an active ingredient for the composition for dentin or dental pulp regeneration.

To be applied to the medicine, the ameloblasts, apical bud cells, or its culture fluid of the present invention can additionally include one or more effective ingredients having the same or similar function to the ameloblasts, apical bud cells, or its culture fluid.

The ameloblasts, apical bud cells, or its culture fluid can be administered orally or parenterally and be used in general forms of pharmaceutical formulation. That is, the ameloblasts, apical bud cells, or its culture fluid of the present invention can be prepared for oral or parenteral administration by mixing with generally used diluents or excipients such as fillers, extenders, binders, wetting agents, disintegrating agents and surfactants. Solid formulations for oral administration are tablets, pills, powders, granules and capsules. These solid formulations are prepared by mixing the ameloblasts, apical bud cells, or its culture fluid of the present invention with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The effective dosage of the ameloblasts, apical bud cells, or its culture fluid of the present invention can be determined according to weight, age, gender, health condition, diet, administration frequency, administration method, excretion and severity of a disease. The dosage is 0.001˜100 mg/kg per day, and preferably 0.1˜10 mg/kg per day, and administration frequency is preferably 1˜6 times a day.

The ameloblasts, apical bud cells, or its culture fluid of the present invention can be administered alone or together with surgical operation, radiotherapy, hormone therapy, chemotherapy and biological regulators.

The present invention also provides a method for promoting hard tissue formation containing the step of administering a pharmaceutically effective dose of ameloblasts, apical bud cells, or its culture fluid to a subject.

The present invention further provides a pharmaceutical composition for promoting hard tissue formation comprising ameloblasts or its culture fluid as an active ingredient.

The ameloblasts herein are preferably ameloblast-lineage cells, but not always limited thereto.

The present invention also provides a pharmaceutical composition for promoting hard tissue formation comprising apical bud cells or its culture fluid as an active ingredient.

The culture fluid herein is preferably the one cultured for 0-10 days from the beginning of differentiation, and more preferably the one cultured for 3-7 days, and most preferably cultured for 3 days.

The apical bud cells culture fluid herein is preferably prepared by the following steps, but not always limited hereto.

1) culturing mouse primary apical bud cells until reaching 90% of confluence;

2) culturing the cells in the medium supplemented with ascorbic acid and J-glycerophosphate;

3) collecting the medium after 3 days in culture;

4) filtering the medium; and

5) precipitating proteins in the medium, followed by eliminating salts therein.

In a preferred embodiment of the present invention, the effect of ameloblasts on the differentiation of osteoblasts was investigated. First, expression levels of Runx2, osterix, type I collagen, ALP and BSP, the genes involved in osteoblast differentiation, were measured by real-time PCR and western blotting. As a result, the expressions were all increased significantly. The effect of ameloblasts on the expressions of type collagen and osterix was also investigated. As a result, the expression levels of type I collagen and osterix were significantly increased in osteoblasts co-cultured with ameloblasts compared to the control (see FIG. 5).

In a preferred embodiment of the present invention, to investigate the effects of apical bud cells culture fluid on hard tissue formation, dental pulp stem cells were mixed with HA/TCP and then transplanted into the subcutaneous tissues of nude mice. As a result, it was confirmed that apical bud cells culture fluid induced hard tissue formation (see FIG. 9).

In a preferred embodiment of the present invention, immunohistochemistry was performed to investigate the characteristics of the hard tissue formed by apical bud cells culture fluid. As result, the hard tissue was confirmed to have bone characteristics by identifying BSP mainly expressed in bones (see FIG. 10).

Therefore, it was confirmed that the ameloblasts, apical bud cells and their culture fluids could increase the expressions of Runx2 and osterix which are essential for osteoblast differentiation, increase the expression of type I collagen which is abundant in extracellular matrix approximately 2.5-fold compared to the control, and increase the expression of ALP approximately 7-fold. Particularly, apical bud cells culture fluid induced hard tissue formation and the hard tissue contained BSP, the matrix protein expressed mainly in bones, confirmed by immunohistochemistry. Thus, the said ameloblasts, apical bud cells, or its culture fluid can be effectively used as an active ingredient for a pharmaceutical composition for promoting hard tissue formation.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Cell Lines and Cell Culture

Three cell lines cultured at 37° C. in a 5% CO₂ humidified atmosphere were used in this invention. MDPC-23, the mouse odontoblast cell line, and MG63, the human osteoblast cell line, were grown and maintained in Dulbecco's Modified Eagle's Medium (Invitrogen) supplemented with 10% heat-inactivated fetal bovine serum. KB carcinoma cells, the human oral epithelial cells, were grown in MEM supplemented with 10% heat-inactivated fetal bovine serum.

### Example 2

### Isolation and Culture of Primary Apical Bud Cells (ABCs) and Human Dental Pulp Stem Cells (hDPSCs)

The lower incisors were separated from anesthetized mice at postnatal day 7. Apical bud was enzymatically isolated therefrom by using a needle. The isolated tissue was cultured in DMEM/F12. Epithelial cells were isolated by treating trypsin at different concentrations. The isolated cells were cultured in keratinocyte serum-free medium (GIBCO-BRL, USA) until reaching confluence. After reaching confluence, the cells were cultured in DMEM supplemented with 10% heat-inactivated FBS.

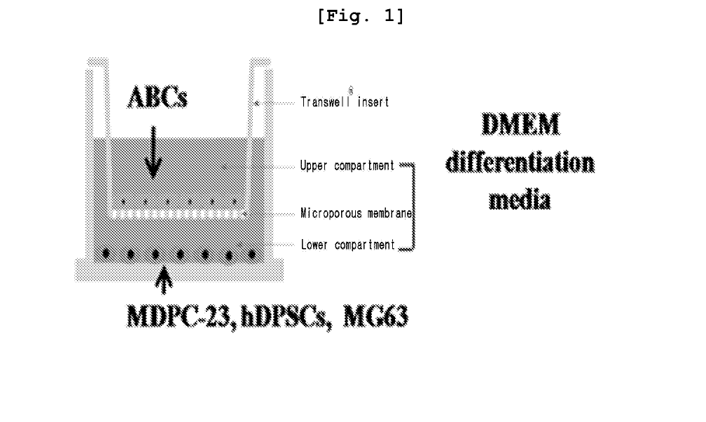

Human dental pulp stem cells were isolated from the wisdom teeth of 10 adults (18-22 years of age) at the Seoul National University Dental Hospital. The experimental protocol was approved by the hospital's Institutional Review Board, and the patients provided informed consent. The isolation was performed according to the method of Jung H S et al (Jung H S, Lee D S, Lee J H, Park S J, Lee G, Seo B M, et al. Directing the differentiation of human dental follicle cells into cementoblasts and/or osteoblasts by a combination of HERS and pulp cells. J Mol. Histol. 2011; 42:227-35.). Briefly, the teeth were cracked and opened, and the pulp tissues were removed gently with forceps, minced into explants, and placed in 60 mm culture dishes. The explants were cultured in DMEM. To culture apical bud cells simultaneously with odontoblasts, dental pulp stem cells or osteoblasts, transwell dishes (3450-clear, Corning, USA) were used. Apical bud cells were cultured in the insert of the transwell dish, and odontoblasts, dental pulp stem cells or osteoblasts were cultured in the base of the dish, respectively. When confluence reached 80-90%, the insert was combined with the base, and differentiation was induced using differentiation medium supplemented with 50 mg/ml ascorbic acid and 10 mM β-glycerophosphate for 10 days (FIG. 1).

### Example 3

### Preparation of Apical Bud Cells Conditioned Medium

Apical bud cells were seeded at the density of 7.5×10⁵ cells on 100 mm dishes. When confluence reached 90%, the cells were cultured in differentiation medium supplemented with 50 mg/ml ascorbic acid and 10 mM 8-glycerophosphate. The day before differentiation was regarded as day 0. On day 0 and day 3, the cells were washed twice with PBS, and differentiation medium without FBS/EGF was added. The conditioned medium was collected three times at 8 hour intervals. After filtration using a 0.2-μm pore filter (Nalgene, USA), 200 ml of harvested conditioned medium was concentrated using ammonium sulfate (80 g) precipitation and dialyzed against PBS at a low temperature to remove salts.

### Example 4

### Differentiation of Apical Bud Cells into Ameloblasts

To confirm the differentiation apical cells into ameloblasts, total RNA was extracted. cDNA was synthesized by using 2 μg of total RNA, 1 ul of reverse transcriptase, and 0.5 μg of oligo (dT). The synthesized cDNA was used for real-time PCR. Real-time PCR was performed on an ABI PRISM 7500 sequence detection system (Applied Biosystems) using SYBR GREEN PCR Master Mix (Takara, Japan). The PCR conditions were 94° C. for 1 min followed by 95° C. for 15 sec and 60° C. for 34 sec for 40 cycles. The relative differences in PCR results were calculated using the comparative cycle threshold (C_{T}) method. Primer sequences are shown in Table 1.

TABLE 1
Genomic DNA PCR

| | | | |
|---|---|---|---|
| Human Alu sequence | Forward | CGA GCG GTG ATC TGA GT | (SEQ ID NO: 1) |
| | Reverse | TCT TCG CCA GCC GAC | (SEQ ID NO: 2) |

RT-PCR

| | | | |
|---|---|---|---|
| Amcloblastin | Forward | AAA AGG AGA AGG TCC AGA AG | (SEQ ID NO: 3) |
| | Reverse | TGC GGA AGG ATA GTA AGT GT | (SEQ ID NO: 4) |
| Amelogenin | Forward | CCA GAG CAT GAT AAG GCA GC | (SEQ ID NO: 5) |
| | Reverse | GAA CTG GCA TCA TTG GTT GC | (SEQ ID NO: 6) |
| Hspa [symbol] | Forward | GGG TAA CCG CAC CAC GCC AA | (SEQ ID NO: 7) |
| | Reverse | TCG AGC CCG GGT AAT GGA GGT | (SEQ ID NO: 8) |
| Lgal [symbol] bp | Forward | GCG CTT GGT TAA CGG GGC CT | (SEQ ID NO: 9) |
| | Reverse | ATG ACG CTG CTG CCC ACC AC | (SEQ ID NO: 10) |
| Hsp90ab1 | Forward | CTT CAT TCC CCG GCG GGC TC | (SEQ ID NO: 11) |
| | Reverse | CTC AGA GAG GCG GCG TCG GT | (SEQ ID NO: 12) |
| GAPDH | Forward | CTC ACA GTC CAT GCC ATC AC | (SEQ ID NO: 13) |
| | Reverse | TCC ACC ACC CTG TTG CTG T | (SEQ ID NO: 14) |

Real time-PCR

| | | | |
|---|---|---|---|
| DSPP | Forward | GTG ACG ACA AGG ACG AAT CTG A | (SEQ ID NO: 15) |
| | Reverse | CAC TAC TGT CAC TGC TGT CAC T | (SEQ ID NO: 16) |
| BSP | Forward | ACC TTT TAT GTC CCC CGT TGA | (SEQ ID NO: 17) |
| | Reverse | TGG ACT GGA AAC CGT TTC AGA | (SEQ ID NO: 18) |
| Nest [symbol] | Forward | CCC TGA AGT CGA GGA GCT G | (SEQ ID NO: 19) |
| | Reverse | CTG CTG CAC CTC TAA GCG A | (SEQ ID NO: 20) |
| Col1 | Forward | GCT CCT CTT AGG GGC CAC | (SEQ ID NO: 21) |
| | Reverse | CCA CGT CTC ACC ATT GGG G | (SEQ ID NO: 22) |
| Ru [symbol] 2 | Forward | CAG GTA CGT GTG GTA GTG AGT | (SEQ ID NO: 23) |
| | Reverse | TTC TCC AAC CCA CGA ATG CAC | (SEQ ID NO: 24) |
| ALP | Forward | CCA ACT CTT TTG TGC CAG AGA | (SEQ ID NO: 25) |
| | Reverse | GGC TAC ATT GGT GTT GAG CTT TT | (SEQ ID NO: 26) |
| OC | Forward | CTG ACA AAG CCT TCA TGT CCA A | (SEQ ID NO: 27) |
| | Reverse | GCG CCG GAG TCT GTT CAC TA | (SEQ ID NO: 28) |
| GAPDH | Forward | AGG TCG GTG TGA ACG GAT TTG | (SEQ ID NO: 29) |
| | Reverse | TGT AGA CCA TGT AGT TGA GGT CA | (SEQ ID NO: 30) |

[symbol]

indicates data missing or illegible when filed

As a result, as shown in FIG. 2, it was confirmed that apical bud cells expressed mRNAs of ameloblast differentiation markers, ameloblastin and amelogenin (FIG. 2).

### Experimental Example 1

### Effect of Apical Bud Cells on Odontoblast Differentiation

To investigate whether apical bud cells induce odontoblast differentiation, the following experiments were performed. DSPP, the noncollageneous protein playing an important role in dentin formation, is regarded as a specific marker of differentiated odontoblasts. So, the present inventors co-cultured odontoblasts with apical bud cells and analyzed the expression of DSPP by real time PCR and western blotting. Particularly, PCR and western blotting were performed as follows:

### <1-1> Promotion of Odontoblast Differentiation Confirmed by Gene Amplification

Total RNA was extracted from odontoblasts, dental pulp stem cells and osteoblasts with TRIzol reagent. cDNA was synthesized by using 2 μg of total RNA, 1 ul of reverse transcriptase, and 0.5 μg of oligo (dT). The synthesized cDNA was used for real-time PCR. Real-time PCR was performed on an ABI PRISM 7500 sequence detection system (Applied Biosystems) using SYBR GREEN PCR Master Mix (Takara, Japan). The PCR conditions were 94° C. for 1 min followed by 95° C. for 15 sec and 60° C. for 34 sec for 40 cycles. The relative differences in PCR results were calculated using the comparative cycle threshold (C_{T}) method. Primer sequences are shown in Table 1.

As a result, as shown in FIG. 3A-FIG. 3C, DSPP expression increased approximately 2-fold (FIG. 3A), and nestin expression increased approximately 4-fold after 10 days in differentiation (FIG. 3B). But, BSP expression was not much changed (FIG. 3C).

### <1-2> Promotion of Odontoblast Differentiation Confirmed by Western Blotting

To isolate protein, cells were washed with PBS, followed by centrifugation at 1,000×g for 5 minutes at a low temperature. After removal of the supernatant, the pellet was resuspended in lysis buffer (100 mM Tris, pH 7.4, 350 mM NaCl, 10% glycerol, 1% Nonidet P-40, 1 mM EDTA, 1 mM dithiothreitol, 1× protease inhibitor) (SigmaAldrich, St. Louis, Mo., USA) and incubated for 15 min on ice. Cell debris was eliminated by centrifugation at 16,000×g for 15 minutes at a low temperature. Then, supernatant was obtained. 30 μg of protein was separated by electrophoresis and transferred onto a PVDF membrane. The membrane was blocked with 5% nonfat dry milk in PBS containing 0.1% Tween 20 (PES-T) and reacted with antibodies against DSP, BSP and type I collagen (1:1000). After washing, the membrane was incubated with anti-rabbit IgG conjugated with horseradish peroxidase (1:5000, Santa Cruz Biotechnology, USA) for 1 hr. Labeled protein bands were detected using an ECL system (enhanced chemiluminescence system) (Amersham Biosciences).

As a result, as shown in FIG. 3D-FIG. 3G, the expression levels of DSP protein increased from day 7 and significantly increased at day 10 compared to control. Similarly to mRNA expression, density was confirmed to be almost doubled. The expression levels of type I collagen protein increased approximately 2-fold in co-cultured cells compared to control from days 3 and decreased slowly thereafter. The expression levels of BSP protein decreased significantly (FIG. 3C-FIG. 3E). In the meantime, the effect of apical bud cells on the proliferation of odontoblasts was investigated. As a result, as shown in FIG. 3D, apical bud cells did not have any significant effect on the proliferation of odontoblasts (FIG. 3D).

### Experimental Example 2

### Effect of Apical Bud Cells on Dental Pulp Stem Cells Differentiation

To investigate whether apical bud cells induce dental pulp stem cells differentiation, dental pulp stem cells were co-cultured with apical bud cells, followed by real time PCR and Western blotting by the same manner as described in <Experimental Example 1>.

As a result, as shown in FIG. 4, the expression levels of DSPP, a marker of differentiated odontoblasts, significantly increased, whereas BSP protein decreased from day 3. Similar results were obtained by Western blotting (FIG. 4).

### Experimental Example 3

### Effect of Apical Bud Cells on Osteoblast Differentiation

The expressions of Runx2, osterix, type I collagen, ALP and BSP were investigated by real time PCR by the same manner as described in <1-1> and Western blotting by the same manner as described in <1-2>.

<3-1> Promotion of osteoblast differentiation confirmed by real time PCR

The expression levels of Runx2 and osterix, the transcription factors playing an essential role in osteoblast differentiation, increased in osteoblasts co-cultured with apical bud cells compared to control (FIGS. 5A and 5B). The expression of type I collagen which is abundant in extracellular matrix of bones increased approximately 2.5-fold compared to control, and the expression of ALP increased approximately 7-fold (FIGS. 5C and 5D). Therefore, it was confirmed that apical bud cells could promote the differentiation of osteoblasts.

### <3-2> Promotion of Osteoblast Differentiation Confirmed by Western Blotting

To investigate whether apical bud cells induce osteoblast differentiation, the expression of BSP, the gene involved in osteoblast differentiation, was investigated by Western blotting.

As a result, as shown in FIG. 5E and FIG. 5F, the expression of the gene was significantly increased from days 3 to 7 (FIG. 5E and FIG. 5F). Therefore, it was confirmed that apical bud cells could promote the differentiation of osteoblasts.

### Experimental Example 4

### Effect of Apical Bud Cells Culture Fluid on DSPP Promoter Activity

### <4-1> Selection of Optimum Apical Bud Cells Culture Fluid

To select the most effective apical bud cells culture fluid for odontoblast differentiation, the effects of various apical bud cells culture fluids (days 0, 3, and 7) on DSPP promoter activity in MDPC-23 cells were investigated by luciferase assay.

As a result, as shown in FIG. 6, DSPP promoter activity was increased in all apical bud cells culture fluids compared to control (MDPC-23 cells alone group). Particularly, apical bud cells culture fluid after 3 days in culture was most effective in increasing DSPP promoter activity, so that the culture fluid from apical bud cells (PA-CM) after 3 days in culture was selected for the following experiment (FIG. 6).

### <4-2> Confirmation of Increased DSPP-Promoter Activity by the Treatment of Apical Bud Cells Culture Fluid

Hereinbefore, it was confirmed that ameloblasts promoted odontoblast differentiation in vitro, and increased the expression of DSPP that was important for dentin formation. In this example, if the substance secreted by apical bud cells could affect DSPP promoter activity was investigated. Various apical bud cells culture fluids (days 0, 3, and 7) were prepared and used for the following experiments. Odontoblasts were seeded in a 24-well plate at the density of 5×10⁴ cells/well. After 24 hours, the cells were transfected with Lipofectamine Plus™ reagent. For each transfection, 0.4 mg of pGL3 basic (control) and DSPP promoter were used. Transfected cells were treated with apical bud cells culture fluid or oral epithelial cells culture fluid for 48 hrS. Then, luciferase activity was measured with luminometer (FLUOStar, Germany).

As a result, as shown in FIG. 6, DSPP promoter activity was increased in all apical bud cells culture fluids (days 0, 3, and 7). Particularly, apical bud cells culture fluid after 3 days in culture was most effective in increasing DSPP promoter activity. When apical bud cells culture fluid was boiled to denature all the proteins, DSPP promoter activity was not increased. This result indicated that the substance in the culture fluid that was able to increase DSPP promoter activity was the protein (FIG. 6A). In the meantime, BSP promoter activity was reduced (FIG. 6B). It was also investigated if the confirmed characteristics above were unique ones of dental epithelium. To do so, KB cells (oral epithelial cells) were used for the same experiment as above. As a result, it was confirmed that KB cells culture fluid had no influence on DSPP promoter activity (FIG. 6).

### Experimental Example 5

### Effect of Apical Bud Cells Culture Fluid on Hard Tissue Formation by Dental Pulp Stem Cells

<5-1> Confirmation of Sternness of Dental Pulp Stem Cells (hDPSCs)

To ascertain the sternness of isolated hDPSCs by explant culture, immunofluorescence staining was performed.

Particularly, human dental pulp stem cells were fixed in 4% paraformaldehyde for 30 min at 4° C. After permeabilization with 0.15% Triton X-100/PBS, nonspecific binding of the antibodies was blocked using 2% bovine serum albumin. Cells were incubated with primary anti-CD44 (550538, BD Pharmigen, San Jose, Calif., USA) and STRO-1 antibodies (MAB1038, R&D Systems, Minneapolis, Minn., USA) for 16 hrS at 4° C. After primary antibody incubation, the cells were washed twice with PBS and incubated with a fluorescent labeled secondary anti-mouse antibody for 1 hr in the dark. DAPI was used to identify cell nuclei. After washing, samples were mounted and analyzed by fluorescent microscopy.

Isolated hDPSCs expressed both STRO-1 and CD-44. As a result, as shown in FIG. 7, it was confirmed that stem cell markers, CD-44 and STRO-1, were expressed (FIG. 7).

### <5-2> Hard Tissue Formation

Dental pulp stem cells are very useful material for dental tissue regeneration based on tissue engineering methods. Dental pulp stem cells are easily isolated from human wisdom teeth and can be differentiated into various cells (Mantesso A, Sharpe P, Dental stem cells for tooth regeneration and repair. Expert Opin Biol Ther. 2009 September; 9(9):1143-54). Many of previous reports showed that hard tissue could be formed by using dental pulp stem cells (Prescott, R. S.; Alsanea, R.; Fayad, M. I.; Johnson, B. R.; Wenckus, C. S.; Hao, J.; John, A. S.; George, A. In vivo generation of dental pulp-like tissue by using dental pulp stem cells, a collagen scaffold, and dentin matrix protein 1 after subcutaneous transplantation in mice. J. Endod., 2008, 34(4), 421-6; Huang, G. T.; Yamaza, T.; Shea, L. D.; Djouad, F.; Kuhn, N. Z.; Tuan, R. S.; Shi, S. Stem/Progenitor cell-mediated de novo regeneration of dental pulp with newly deposited continuous layer of dentin in an in vivo model. Tissue Eng Part A., 2010, 16(2), 605-15). Considering that apical bud cells culture fluid induced odontoblast differentiation, the present inventors tried to evaluate its effect in vivo. Dental pulp stem cells treated with or without concentrated apical bud cells culture fluid were mixed with HA/TCP, which were transplanted into the subcutaneous tissues of nude-mice. Twelve weeks later, the tissues were histologically observed. BMP-2 was used as a positive control, because it is well known that BMP-2 induces hard tissue formation of dental pulp stem cells.

As a result, as shown in FIG. 8, it was confirmed by H-E staining with the isolated, decalcified tissue sections that hard tissue formation was induced in the group treated with apical bud cells culture fluid (FIG. 8). Twelve weeks after transplantation, all groups generated hard tissues on the border of HA/TCP. When areas of formed hard tissue of each group were measured, BMP-2 induced the most hard tissue formation 6 weeks after transplantation. However, 12 weeks after transplantation, the apical bud cells culture fluid-treated group formed similar amounts of hard tissues compared to the BMP-2-treated group (FIGS. 8A-8F). When areas of formed mineralized tissue of each group were measured, BMP-2 induced the most hard tissue formation 6 weeks after transplantation, and apical bud cells culture fluid formed approximately one-half the levels of hard tissue compared to BMP-2. However, 12 weeks after transplantation, the apical bud cells culture fluid-treated group formed similar amounts of hard tissues compared to the BMP-2-treated group (FIG. 8G).

<5-3> Alizarin red S staining

To compare calcification, calcium accumulation was investigated by alizarin red S staining. Odontoblasts were seeded on 35-mm dishes and cultured in differentiation media for 2 weeks with or without apical bud cells culture fluid, followed by alizarin red S staining. On day 0, day 3, day 7, and day 10, each dish was washed three times with PBS and fixed in 4% paraformaldehyde (PFA). After fixing, the cells were washed three times with water, and stained with a 1% alizarin solution (Sigma Aldrich, St. Louis, Mo., USA) for 20 minutes. The excessive staining solution was washed off with water. The stained part was photographed by optical microscope and the density of the part was measured.

As shown in FIG. 6C, calcium accumulation was investigated after treating odontoblasts with the concentrated apical bud cells culture fluid after 3 days in differentiation. As a result, the differentiation was more accelerated and thus calcium accumulation level was higher on the 14^{th} day of differentiation.

### Experimental Example 6

### Mouse In Vivo Transplantation and Histological Analysis

<6-1> Promotion of Differentiation of Dental Pulp Stem Cells into Odontoblasts by Treatment of Apical Bud Cells Culture Fluid

To investigate if the treatment of apical bud cells culture fluid could promote the differentiation of dental pulp stem cells to odontoblasts, dental pulp stem cells (1.0×10⁷ cells) were mixed with 100 mg hydroxyapatite/tricalcium phosphate (HA/TCP) ceramic powder (Zimmer Inc., Warsaw, Ind., USA) with or without 50 μg of apical bud cells culture fluid (PA-CM) and were then transplanted subcutaneously into nude-mice (NIH-bg-nu/nu-xid, Harlan Sprague Dawley, Indianapolis, Ind., USA). Samples were obtained after 6 and 12 weeks, respectively, fixed in 4% paraformaldehyde, and decalcified in a 10% ethylenediaminetetra-acetic acid (EDTA, pH 7.4) solution for a week. The samples were washed, dehydrated, and embedded in paraffin. The paraffin block was sectioned in 5 μm thickness, and the sections were stained with hematoxylin-eosin (H-E). For immunohistochemistry, the sections proceeded to deparaffin and hydration, followed by reaction with 0.6% H₂O₂/methanol for 20 minutes. After blocking with PBS containing 1% bovine serum albumin, the sections were reacted with DSP, BSP and human nuclei antibodies (1:100) at a low temperature. Secondary anti-rabbit IgG antibodies were incubated with the sections at room temperature for 30 min, which were then reacted with the avidin-biotin-peroxidase complex (Vector Laboratory, Burlingame, Canada). Signals were converted using a DAB kit (Vector Laboratory, Burlingame, Canada). The sections were washed, dehydrated and sealed, followed by observation under optical microscope. For scanning electron microscopy (SEM), samples were fixed in 0.1 M cacodylate buffer (pH 7.3) containing 2.5% glutaraldehyde for 30 min and in 0.1 M cacodylate buffer (pH 7.4) containing 1% osmium tetroxide for 1 hr. After rapid dehydration through an ethanol gradient, critical point drying, and sputter coating with gold, cells were observed by SEM (S-4700, HITACHI, Tokyo, Japan).

As a result, as shown in FIG. 9, odontoblasts were formed as a palisade layer at the pulp-dentin interface and so was mineralized dentin. Morphologically, hDPSCs generated a dentin/pulp-like structure lined with odontoblast-like cells that surround a pulp-like tissue in the hDPSCs only group (FIG. 9A). There are pulp-like structures in all groups. However, dentinal tubule structure was hardly observed in odontoblast-like formed in the hDPSCs only and rhBMP-2-treated groups. In addition, a few cells in the rhBMP-2-treated group were entrapped in the mineralized matrix, possessing osteocyte characteristics (FIGS. 9A and 9B). However, the apical bud cells culture fluid-treated group showed odontoblasts like character that the cells exhibited odontoblast characteristics with palisade arrangement along the pulp and typical odontoblast processes in the dentin matrix (FIG. 9C). By SEM, the odontoblasts-like cells appeared elongated and had odontoblast processes (FIG. 9D). These results suggest that in contrast to the hDPSCs only group, dentin/pulp-like complex formation was more extensive in the apical bud cells culture fluid-treated group and the histological architecture of the tissue generated in the apical bud cells culture fluid-treated group resembles the morphology of the normal dentin-pulp complex more closely than tissues generated in the other groups (FIG. 9).

### <6-2> Characteristics of Newly Formed Hard Tissue by Human Dental Pulp Stem Cells

To investigate the characteristics of hard tissue formed similarly to dentin-pulp complex, immunohistochemistry was used by the same manner as described in Example <6-1>. Anti-human nuclei antibody was used to confirm the origin of the hard tissue.

BSP and DSP are regarded as specific markers of osteoblast and odontoblast differentiation. DSP expression was observed weakly in the dental pulp stem cells only group (FIG. 10A), but barely in the rhBMP-2-treated group (FIG. 10B). However, in the apical bud cells culture fluid-treated group, the expression of DSP protein was clearly detected in odontoblast-like cells that contact the formed matrix (FIG. 10C). In the dental pulp stem cells only (FIG. 10D) and apical bud cells culture fluid-treated groups, BSP was weakly expressed (FIG. 10F), whereas in the rhBMP-2-treated group, BSP was detected strongly in both the cells and matrix (FIG. 10E).

However, negative control showed negative staining (FIG. 10G). These findings indicate that the cells in the apical bud cells culture fluid-treated group resemble the characteristics of normal odontoblasts more closely than other groups and that consequently apical bud cells culture fluid induces the odontoblast differentiation of hDPSCs in vivo. In the meantime, the formed hard tissue was specifically stained with anti-human nuclei antibody (FIG. 10H), and the human-specific Alu sequence was amplified (FIG. 10I). These results suggest that the hard tissue is formed by human dental pulp stem cells.

### Experimental Example 7

### Dental Pulp Tissue Regeneration of Dental Pulp Stem Cells Induced by Apical Bud Cells Culture Fluid

Apical bud cells culture fluid was treated to the general tooth-like environment where dental pulp exists. Thereafter, tissue formation was investigated.

As a result, as shown in FIG. 11, pulp-like tissue was not formed when dental pulp stem cells only group was placed in tooth slices, the tooth-like environment, but skeletal muscle was formed therein (FIG. 11). In the dental pulp stem cells only group, the inside of tooth slice was almost filled with skeletal muscle, and had less number of dental pulp stem cells in dentin layer than in the apical bud cells culture fluid-treated group. Most cells in the dental pulp stem cells only group arranged on the surface of dentin were flat and showed different morphology from odontoblasts (FIGS. 11A-11D). On the other hand, when dental pulp stem cells treated with apical bud cells culture fluid were placed in tooth slices, pulp-like structure was formed therein. In the apical bud cells culture fluid-treated group, the number of dental pulp stem cells arranged in dentin was higher than that in the dental pulp stem cells only group, and dentinal tubule-like structure usually shown in differentiated odontoblasts was also observed (FIGS. 11E-11H).

### Experimental Example 8

### Characteristics of Dental Pulp Tissue Formed in Tooth Environment

After eliminating dental pulp completely, human tooth was sliced, followed by sterilization. The sterilized tooth slices were treated with dental pulp stem cells (5.0×10⁵) and with or without apical bud cells culture fluid (8 ug), which were transplanted subcutaneously into nude-mice (NIH-bg-nu/nu-xid, Harlan Sprague Dawley, Indianapolis, Ind., USA). Samples were obtained after 12 weeks, fixed in 4% paraformaldehyde, and decalcified in a 10% ethylenediaminetetra-acetic acid (EDTA, pH 7.4) solution for a week. The samples were washed, dehydrated, and embedded in paraffin. The paraffin block was sectioned in 5 um thickness, and the sections were stained with hematoxylin-eosin (H-E). To confirm histological characteristics, immunohistochemistry was performed using nestin antibody expressed in dentinal tubule and human specific h-nuclei antigen.

As a result, as shown in FIG. 12, nestin expression was hardly observed in the dental pulp stem cells only group (FIG. 12A). In the meantime, nestin expression was observed between dentin and the cells arranged in the border of dentin in the apical bud cells culture fluid-treated group (FIG. 12B). Besides, tissues were stained by human specific antigen, suggesting that hard tissue was formed by human dental pulp stem cells (FIG. 12C and FIG. 19D).

As shown in FIG. 13, in the group where dental pulp stem cells were placed in tooth slices, it was observed by SEM that dentinal tubule structure was formed only in one or two cells. The observed dentinal tubule structure was not found in the inside of tooth slice. In the meantime, the histological architecture of the tissue generated in the apical bud cells culture fluid-treated group resembles the morphology of normal dentinal tubule structures more closely than in control. Most of such dentinal tubule structure was in the inside of tooth slice. In the apical bud cells culture fluid-treated group, there were lots of dental pulp stem cells arranged with dentin. Therefore, it was confirmed that apical bud cells culture fluid induced odontoblast differentiation of dental pulp stem cells (FIG. 13).

### Experimental Example 9

### Origin of Dental Pulp Stem Cells

To investigate whether the newly formed hard tissue was originated from human dental pulp stem cells, genomic DNA was extracted from the cells isolated from sectioned slices. Human Alu sequence was amplified by using 2 ul of the extracted genomic DNA. As a positive control, 1.0 mg/mL human DNA was used, and 1.0 mg/mL nude mouse genomic DNA was used as a negative control. Human Alu sequence was amplified by PCR as follows; predenaturation at 94□ for 5 minutes, denaturation at 94° C. for 30 seconds, annealing at 58° C. for 30 seconds, polymerization at 72° C. for 45 seconds, 35 cycles from denaturation to polymerization. The PCR products were confirmed by electrophoresis performed on a 2% agarose gel. Primer sequences are shown in Table 1.

As a result, as shown in FIG. 10I, it was confirmed that human Alu sequence was amplified, therefore suggesting that the newly formed hard tissue is originated from human dental pulp stem cells (FIG. 10I).

### Experimental Example 10

### Proteomic Analysis of Apical Bud Cells Conditioned Medium

To confirm whether the promotion effect of apical bud cells conditioned medium on odontoblast differentiation and hard tissue formation was attributed to BMP (bone morphogenetic protein) having the effect of promoting the differentiation of dental pulp stem cells and hard tissue formation, the present inventors blocked BMP activity in conditioned medium with a BMP antagonist, noggin and evaluated DSPP promoter activity.

As a result, there were no changes in DSPP promoter activity after the BMP antagonist treatment, suggesting that conditioned medium affects DSPP promoter activity by factors that are irrelevant to BMP (FIG. 14A). Apical bud cells culture fluids (day 0, and day 3) proceeded to electrophoresis with SDS-PAGE, followed by staining with Coomassie blue. After reducing with DTT, alkylating with 500 mM iodoacetamide, and desalting with NAPS column, the sample was separated by protein electrophoresis, followed by confirmation with Coomassie blue staining. The confirmed sample was treated with trypsin and cut to peptides. The sample was analyzed with a mass spectrometer, and proteins were identified. ProtAn X program was used for the protein identification.

As a result, as shown in FIG. 15, total 113 proteins were confirmed by LC-MSMS analysis, and among them, 23 proteins were known to co-exist on day 0 and day 3 (FIG. 15A and Table 2). The confirmed proteins were classified by their locations. Because of protein localization analysis, 18% of proteins were found to exist in the cytoplasm and 15% in the extracellular matrix (FIG. 15B). In the aspect of functions, the proteins involved in the regulation of metabolic process were the majority and next were the proteins involved in cell growth (FIG. 15C). To confirm if those proteins existed in ameloblasts, RT-PCR was performed with some selected genes. As a result, it was confirmed that those proteins were the ones existing in ameloblasts. Interestingly, periostin was identified. Periostin is expressed first in the teeth and periodontal ligament and is known to take part in integrity maintenance of the periodontal ligament (FIG. 15D) (Horiuchi K, Amizuka N, Takeshita S, Takamatsu H, Katsuura M, Ozawa H, Toyama Y, Bonewald L F, Kudo A., Identification and characterization of a novel protein, periostin, with restricted expression to periosteum and periodontal ligament and increased expression by transforming growth factor beta., J Bone Miner Res. 1999 July; 14(7):1239-49.).

TABLE 2

| | Day 0 | | | |
|---|---|---|---|---|
| | CM | Day 3 | | |
| | Num | CM Num | Num | Num |
| Gene Name | peps_0 | peps_3 | peps_sub | peps_ratio |
| Cpne7/Copine 7 | 1 | 4 | 3 | 4.0 |
| Ttn/Isoform 1 of Titin | 1 | 3 | 2 | 3.0 |
| Hspa8/Heat shock cognate | 9 | 23 | 14 | 2.6 |
| 71 kDa protein | | | | |
| Actn1/Alpha-actinin-1 | 2 | 4 | 2 | 2.0 |
| Pcolce/53 kDa protein | 6 | 10 | 4 | 1.7 |
| Psma [symbol] /Proteasome | 3 | 5 | 2 | 1.7 |
| subunit alpha type-1 | | | | |
| Col3a1/Collagen alpha- [symbol] | 12 | 19 | 7 | 1.6 |
| chain | | | | |
| [symbol] OC100047236/ | 7 | 10 | 3 | 1.4 |
| similar to 14-3-3 zeta | | | | |
| Tkt/Transketolase | 4 | 5 | 1 | 1.3 |
| Ywhae/14-3-3 protein | 12 | 13 | 1 | 1.1 |
| epsilon | | | | |
| Actb/Actin (beta) | 45 | 47 | 2 | 1.0 |
| EG432987/Predicted | 23 | 23 | 0 | 1.0 |
| Ald [symbol] /Fructose- | 4 | 4 | 0 | 1.0 |
| bisphosphate aldolase | | | | |
| [symbol] gals3bp/Putative | 2 | 2 | 0 | 1.0 |
| uncharacterized protein | | | | |
| Hsp90ab1/heat shock | 24 | 23 | −1 | 1.0 |
| protein 90 alpha (cytosolic) | | | | |
| Ywhab/Isoform Long of | 7 | 6 | −1 | 0.9 |
| 14-3-3 protein beta (alpha) | | | | |
| Fn [symbol] /Fibronectin | 115 | 90 | −25 | 6.8 |
| Postn/Isoform 3 of Periostin | 16 | 9 | −7 | 0.6 |
| T [symbol] bs1/Thrombosporidin 1 | 21 | 11 | −10 | 0.5 |
| [symbol] mna/Isoform A of | 10 | 4 | −6 | 0.4 |
| Lamin-A/C | | | | |
| CO11a1/Isoform 1 of | 60 | 22 | −38 | 0.4 |
| Collagen alpha- [symbol] chain | | | | |
| Eef2/Elongation factor 2 | 25 | 9 | −16 | 0.4 |
| Pkm2/Isoform M2 of | 17 | 6 | −11 | 0.4 |
| Pyruvate kinase isozymes | | | | |
| M1/M2 | | | | |

[symbol]

indicates data missing or illegible when filed

### Experimental Example 11

### Effect of Periostin on Hard Tissue Formation

Periostin was identified in Example 10. Thus, it was further investigated if periostin played any part in apical bud cells culture fluid. The effect of periostin on odontoblast differentiation was investigated by using periostin-neutralized conditioned medium (PNSF).

As a result, as shown in FIG. 14B, DSP protein was slightly increased in the periostin-neutralized conditioned medium (PNSF) during odontoblast culture, suggesting that odontoblasat differentiation is promoted by substances in apical bud cells culture fluid rather than periostin (FIG. 14B).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for dentin or dental pulp regeneration containing the step of administering a pharmaceutically effective dose of ameloblasts, apical bud cells, or its culture fluid to a subject.

2. The method for dentin or dental pulp regeneration according to claim 1, wherein the ameloblasts are ameloblast-lineage.

3. The method for dentin or dental pulp regeneration according to claim 1, wherein the culture fluid is prepared by culturing apical bud cells for 3-7 days.

4. The method for dentin or dental pulp regeneration according to claim 1, wherein the administration is performed to dentin or dental pulp.

5. A method for inducing hard tissue formation containing the step of administering a pharmaceutically effective dose of ameloblasts, apical bud cells, or its culture fluid to a subject.

6. The method for inducing hard tissue formation according to claim 5, wherein the ameloblasts are ameloblast-lineage.

7. The method for inducing hard tissue formation according to claim 5, wherein the culture fluid is prepared by culturing apical bud cells for 3-7 days.

8. The method for inducing hard tissue formation according to claim 5, wherein the administration is performed to hard tissue.

9. A pharmaceutical composition for dentin or dental pulp regeneration comprising ameloblasts, apical bud cells, or its culture fluid as an active ingredient.

10. A pharmaceutical composition for inducing hard tissue formation comprising ameloblasts, apical bud cells, or its culture fluid as an active ingredient.


SEQUENCE LISTING (30 sequences)

SEQ ID NO: 1 - DNA, 17 bp, Artificial Sequence
  Human Alu forward sequence
cgagcggtgatctgagt

SEQ ID NO: 2 - DNA, 15 bp, Artificial Sequence
  Human Alu reverse sequence
tcttcgccagccgac

SEQ ID NO: 3 - DNA, 20 bp, Artificial Sequence
  Ameloblastin forward primer
aaaaggagaaggtccagaag

SEQ ID NO: 4 - DNA, 20 bp, Artificial Sequence
  Ameloblastin reverse primer
tgcggaaggatagtaagtgt

SEQ ID NO: 5 - DNA, 20 bp, Artificial Sequence
  Amelogenin forward primer
ccagagcatgataaggcagc

SEQ ID NO: 6 - DNA, 20 bp, Artificial Sequence
  Amelogenin reverse primer
gaactggcatcattggttgc

SEQ ID NO: 7 - DNA, 20 bp, Artificial Sequence
  Hspa8 forward primer
gggtaaccgcaccacgccaa

SEQ ID NO: 8 - DNA, 21 bp, Artificial Sequence
  Hspa8 reverse primer
tcgagcccgggtaatggaggt

SEQ ID NO: 9 - DNA, 20 bp, Artificial Sequence
  Lgals3bp forward primer
gcgcttggttaacggggcct

SEQ ID NO: 10 - DNA, 20 bp, Artificial Sequence
  Lgals3bp reverse primer
atgacgctgctgcccaccac

SEQ ID NO: 11 - DNA, 20 bp, Artificial Sequence
  Hsp90ab1 forward primer
cttcattccccggcgggctc

SEQ ID NO: 12 - DNA, 20 bp, Artificial Sequence
  Hsp90ab1 reverse primer
ctcagagaggcggcgtcggt

SEQ ID NO: 13 - DNA, 20 bp, Artificial Sequence
  GAPDH forward primer
accacagtccatgccatcac

SEQ ID NO: 14 - DNA, 19 bp, Artificial Sequence
  GAPDH reverse primer
tccaccaccctgttgctgt

SEQ ID NO: 15 - DNA, 22 bp, Artificial Sequence
  DSPP forward primer
gtgaggacaaggacgaatgtga

SEQ ID NO: 16 - DNA, 22 bp, Artificial Sequence
  DSPP reverse primer
cactactgtcactgctgtcact

SEQ ID NO: 17 - DNA, 21 bp, Artificial Sequence
  BSP forward primer
aacttttatgtcccccgttga

SEQ ID NO: 18 - DNA, 21 bp, Artificial Sequence
  BSP reverse primer
tggactggaaaccgtttcaga

SEQ ID NO: 19 - DNA, 19 bp, Artificial Sequence
  Nestin forward primer
ccctgaagtcgaggagctg

SEQ ID NO: 20 - DNA, 19 bp, Artificial Sequence
  Nestin reverse primer
ctgctgcacctctaagcga

SEQ ID NO: 21 - DNA, 19 bp, Artificial Sequence
  Col I forward primer
gctcctcttaggggccact

SEQ ID NO: 22 - DNA, 19 bp, Artificial Sequence
  Col I reverse primer
ccacgtctcaccattgggg

SEQ ID NO: 23 - DNA, 21 bp, Artificial Sequence
  Runx2 forward primer
caggtacgtgtggtagtgagt

SEQ ID NO: 24 - DNA, 21 bp, Artificial Sequence
  Runx2 reverse primer
ttctccaacccacgaatgcac

SEQ ID NO: 25 - DNA, 21 bp, Artificial Sequence
  ALP forward primer
ccaactcttttgtgccagaga

SEQ ID NO: 26 - DNA, 23 bp, Artificial Sequence
  ALP reverse primer
ggctacattggtgttgagctttt

SEQ ID NO: 27 - DNA, 22 bp, Artificial Sequence
  OC forward primer
ctgacaaagccttcatgtccaa

SEQ ID NO: 28 - DNA, 20 bp, Artificial Sequence
  OC reverse primer
gcgccggagtctgttcacta

SEQ ID NO: 29 - DNA, 21 bp, Artificial Sequence
  GAPDH forward primer
aggtcggtgtgaacggatttg

SEQ ID NO: 30 - DNA, 23 bp, Artificial Sequence
  GAPDH reverse primer
tgtagaccatgtagttgaggtca